(19) United States
(12) Patent Application Publication
(10) Pub. No.: US 2005/0112068 A1
(43) Pub. Date: May 26, 2005

(54) Systems and methods for reducing unintended use of active ingredients in dermal delivery devices

(76) Inventors: Kevin S. Warner, West Jordan, UT (US); Michael A. Ashburn, Salt Lake City, UT (US); Suyi Niu, Salt Lake City, UT (US)

Correspondence Address: THORPE NORTH & WESTERN, LLP, P.O. Box 1219, Sandy, UT, 84091-1219, US

(21) Appl. No.: 10/975,766

(22) Filed: Oct. 27, 2004

Related U.S. Application Data
(60) Provisional application No. 60/515,026, filed on Oct. 28, 2003.

(51) Int. Cl. A61K 49/00; A61K 9/70

(52) U.S. Cl. 424/10.1; 424/449

(57) ABSTRACT

The present invention is drawn to systems and methods for reducing unintended use of an active ingredient, such as residual active ingredients present in spent dermal patches and peels. The system can include a dermal patch including an active ingredient and a destructive agent configured to chemically react with the active ingredient. The destructive agent can be present in a container or an absorber. In one embodiment, the absorber can be used within the container. The container can be configured to receive the dermal patch such that the active ingredient contacts the destructive agent within the container. The absorber can be configured to contact the dermal patch such that the active ingredient contacts the destructive agent of the absorber. Additionally, systems and methods for impeding the unintended use of an active ingredient, such as those present in dermal patches or peels, are also provided. The system includes a dermal patch having a first side configured to deliver an active ingredient to a skin or mucosal surface, wherein the first side also includes a dermal adhesive. The system also includes an adhesive-coated device. The adhesive-coated surface of the device can be configured to adhere to the first side, wherein upon use of the dermal patch followed by contacting the adhesive-coated surface with the first side, residual active ingredient is rendered substantially inaccessible.

The present application claims the benefit of U.S. Provisional Application No. 60/515,026 filed Oct. 28, 2003, which is incorporated herein by reference.

### FIELD OF THE INVENTION

The present invention is related to the disposal of dermal patches or peels, including transdermal patches, after use. More particularly, the present invention is drawn to the destruction or impeding of unintended use of residual drug substances that typically remains in dermal patches or peels after intended use.

### BACKGROUND OF THE INVENTION

Dermal patches for topical treatment of the skin, regional treatment of tissue, or systemic treatment via the circulatory system have been used for many purposes. One type of dermal patch that can be used is for topical or regional tissue treatment. Another type is the transdermal patch which facilitates the transfer of an active agent through the derma for systemic uptake. Generally, dermal patches, including transdermal patches, can be prepared for skin adhesion, or can be prepared for mucosal tissue adhesion. Two common types of patches currently used in the marketplace include reservoir patches and matrix patches. Additionally, dermal peels are also used to deliver drugs.

Transdermal products have become an important part of drug delivery and treatment of various tissue specific or systemic ailments or conditions. Currently there are a handful of active ingredients or drugs available as transdermal patches, with new products being developed continuously. Examples of drugs administered transdermally and their associated treatments include: scopolamine for treatment of motion sickness, nitroglycerin or isosorbide dinitrate for treatment of chest pain or discomfort due to coronary heart disease, estradiol and/or other hormones for relief of menopausal symptoms and birth control, clonidine for treatment of high blood pressure, fentanyl for treatment of chronic pain, nicotine for smoking cessation, and estradiol/norethisterone acetate or testosterone for hormone replacement therapy. Other patches have been prepared for such indications as birth control.

Currently, used or spent dermal patches that include residual drug are typically thrown into the trash or flushed into the sewage system. These disposal methods pose serious problems including the potential for drug abuse, unintended use, and environmental hazard. For example, a transdermal fentanyl patch that includes residual drug after use can easily be obtained from the trash by a person who wants to extract the residual drug for illegal use or resale. Used dermal patches can also be accessible by small children who live with a patient using a dermal patch for a legitimate medical purpose. Careless disposal can result in the child chewing or swallowing the spent dermal patch, and thus, expose the child to serious risk. Other active ingredients may pollute the environment if they eventually enter the ground water system via landfills or sewage systems. For example, it is a concern that estrogen in millions of disposed dermal patches may enter the water systems that humans and animals use.

In recognition of this problem, it would be desirable to provide systems and methods for proper disposal of dermal patches or peels, including transdermal patches, in order to avoid unintended use and/or environmental damage, e.g., accidental ingestion, pollution of ground water, addicts extracting remaining drug(s) from discarded patches, etc.

### SUMMARY OF THE INVENTION

It has been recognized that it would be advantageous to develop systems and methods for treating or rendering at least one residual active ingredient or drug that remains present in spent dermal patches or peels inoperative, inaccessible, or destroyed. As such, a system for reducing unintended use of an active ingredient can comprise a dermal patch or peel including the active ingredient; a destructive agent that is chemically reactive with the active ingredient; and a device for supporting the destructive agent. The device can be configured to receive the dermal patch or peel and cause the active ingredient to contact the destructive agent.

In another embodiment, a system for reducing unintended use of an active ingredient can comprise a dermal patch or peel including the active ingredient; a destructive agent that is chemically reactive with the active ingredient; and an absorber having the destructive agent impregnated therein. The absorber can be further configured to contact the dermal patch or peel and cause the active ingredient to contact the destructive agent.

Alternatively, a method for destroying an active ingredient in a dermal drug delivery device can comprise steps of selecting a destructive agent that is chemically reactive with the active ingredient of a dermal patch or peel; and contacting the destructive agent with the active ingredient, and thus deactivating the active ingredient.

In another embodiment, a system for impeding the unintended use of an active ingredient can comprise a dermal patch including a first side configured to deliver the active ingredient to a skin or mucosal surface, and a device including a glue-coated surface. The first side of the dermal patch can also include a dermal adhesive, and the glue-coated surface of the device can be configured to adhere to the first side. Thus, upon contacting the glue-coated surface with the first side, residual active ingredient can be rendered substantially inaccessible.

In yet another embodiment, a method for impeding the unintended use of an active ingredient can comprise steps of obtaining a dermal patch including first side configured to deliver the active ingredient to a skin or mucosal surface, and adhering the first side of the spent dermal patch to a glue-coated surface of a device, thereby rendering residual active ingredient substantially inaccessible. In one embodiment, the first side of the dermal patch includes a dermal adhesive.

In another embodiment, a system for impeding unintended use of an active ingredient can comprise a dermal patch or peel configured to deliver the active ingredient to a skin or mucosal surface; and a device configured to envelop and seal the dermal patch or peel therein such that the active ingredient is rendered substantially inaccessible for unintended use.

In a related embodiment, a method for impeding the unintended use of residual active ingredients can comprise steps of obtaining a dermal patch or peel configured to deliver the active ingredient to a skin or mucosal surface; and sealing the dermal patch or peel within the device, thereby rendering the active ingredient substantially inaccessible for unintended use.

Additional features and advantages of the invention will be apparent from the following detailed description which illustrates, by way of example, features of the invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only, and is not intended to be limiting, as the scope of the present invention will be defined only by the appended claims and equivalents thereof.

In describing and claiming the present invention, the following terminology will be used.

The singular forms “a,” “an,” and, “the” include plural referents unless the context clearly dictates otherwise. Thus, for example, reference to “an active ingredient” includes reference to one or more of such active ingredients.

As used herein, the terms “drug” and “active ingredient” can be used interchangeably and refers to substances present in dermal patches or peels that are configured to be delivered either topically, locally, or systemically to a patient. It is often this substance that is desired to be denatured, destroyed, or sealed-off in accordance with embodiments of the present invention.

The terms “dermal” and “dermal delivery” refer to the route of administration of an active ingredient, e.g., a drug delivered to, into, or through the skin or mucosal surface, such as with a matrix patch, a reservoir patch, or a dermal peel. Thus, application of a topical active ingredient, such as an antibiotic or ointment to an injury or burn, or the application of a drug into or through the skin for transdermal delivery is included by the term “dermal delivery.”

The terms “transdermal” and “transdermal delivery” refer to dermal application of an active ingredient or drug, such as with a matrix patch, reservoir patch, or dermal peel, which drug can be transferred into or through the skin or mucosal surfaces for local tissue or systemic administration. Various methods of enhancing transdermal administration include the use of a chemical permeation enhancer(s), the use of heat, the use of an electric field, or the like.

The terms “dermal patch,” “dermal drug patch,” “dermal drug delivery device,” or the like, refer to drug delivery systems for delivering drugs onto the surface of the skin, into the skin, and/or across the skin into regional tissues or systemic circulation. The active ingredient or drug used may be topically, regionally, and/or systemically targeted.

The term “reservoir patch” typically includes a compartment containing a drug solution, gel, or suspension within or on a patch. In one type of reservoir patch, the reservoir of drug can be separated from a release liner by a semi-permeable membrane and a dermal adhesive, wherein the dermal adhesive is used for adhering the patch to the skin or mucosal surface.

The term “matrix patch” typically includes a gel-like, semisolid, or solid polymer matrix containing a drug solution or suspension within or on a patch. The drug is typically in direct contact with a release liner. A dermal adhesive is typically also present to adhere the patch to the skin or mucosal surface. In one embodiment, the drug can be intimately admixed with the dermal adhesive.

The term “dermal peel” or “peel” refers to a semisolid formulation, such as a gel or a cream, that can be applied to skin or mucosa as a thin layer, and which becomes a solidified, flexible, and peelable layer after at least one solvent, such as a volatile solvent, is evaporated. Solidification can occur as a result of drying or evaporation of certain solvents from liquid. After use, the peel can be removed, as the name suggests, by a peeling process. As with dermal patches, often, peels will contain residual amounts of active ingredient or drug, which can be susceptible to intentional abuse or unintentional misuse. For example, if a peel is not properly disposed, a child might obtain the spent dermal peel and place it in his or her mouth.

The term “permeation enhancer” refers to a chemical agent that can be used with a drug present in a patch to increase the rate at which a drug permeates across or into the skin or mucosal surfaces.

The term “destructive agent” refers to a chemical or physical agent(s) capable of chemically or physically destroying a drug in a dermal patch. Destructive chemicals, light, and/or heat are examples of destructive agents that can be used. When referring to chemical destructive agents, often, a solvent is present with the destructive agent that can provide beneficial properties with respect to the destructive agent.

The terms “destructive solution,” “destructive agent-containing solution,” and “destructive agent solution” refer to solutions comprising a solvent and a destructive agent. Typically, the destructive agent is fully solubilized by the solvent, though saturated solutions can also be used.

The term “substantially inaccessible” is used to describe embodiments wherein an adhesive-coated device is used to adhere to a spent dermal patch (or envelop a spent dermal patch or peel) to deter or prevent unintended use, or provide reasonable environmental protection in preparation for disposal. Substantially inaccessible does not infer that the active ingredient would be completely inaccessible, as some tools can be used to override this deterrent effect, e.g., syringes, cutting devices, etc. Thus, by “substantially inaccessible,” what is meant is that by means such as tearing, peeling, shearing, screwing, twisting, or the like, the active ingredient would be more difficult to access.

The term “residual” when referring to an active ingredient refers to an amount of drug or active ingredient that may remain within a dermal patch or peel after use. For example, as it is sometimes difficult for a drug to pass through the skin using a transdermal patch or peel, excess drug is included in the patch matrix, reservoir, or peel in order maintain a sufficient permeation driving force throughout an intended drug administration period. As such, by design, excess drug can be included in a transdermal patch or peel that will remain, even after the dermal patch or peel has been used for an intended amount of time. Thus, for purposes of the present invention, the term residual is not meant to infer that only trace amounts of active ingredient remain. Often, relatively large amounts of active ingredient remain in a dermal patch or peel after use, which amounts would be considered “residual” in accordance with the present definition.

The term “unintended use” refers to uses or occurrences unrelated to product design. For example, accidental ingestion, pollution of ground water, and even intentional extraction of residual drug from discarded patches or peels for illegal drug use are all considered unintended use.

The term “spent” when referring to dermal patches or peels does not mean that the entire active ingredient has been removed. It merely means that the patch or peel has been used for at least a short amount of time, and for one reason or another, is in a condition to be removed or discarded. Residual active ingredient typically remains in a spent dermal patch or peel.

The term “adhesive” can be used in several contexts. A “dermal adhesive” refers to the adhesive that is present on a dermal patch for adhering the dermal patch to skin or a mucosal surface. Conversely, the terms “adhesive-coated surface,” “adhesive-coated device,” and “adhesive-coated impermeable device,” and the like, refer to an adhesive that is present on a substrate that is not used for dermal adhesion, but rather, is used in embodiments where the device is adhered to a spent dermal patch to inhibit unintended use of the patch. Dermal peels are also known to be adhesive with respect to skin or mucosal surfaces to which they are applied.

In accordance with these definitions, a system for reducing unintended use of an active ingredient can comprise a dermal patch or peel including the active ingredient; a destructive agent that is chemically reactive with the active ingredient; and a device for supporting the destructive agent. The device can be configured to receive the dermal patch or peel and cause the active ingredient to contact the destructive agent. In one embodiment, the device can be a container that contains the destructive agent, and further, can be configured to receive the dermal patch or peel such that the active ingredient contacts the destructive agent within the container.

In another embodiment, a system for reducing unintended use of an active ingredient can comprise a dermal patch or peel including the active ingredient; a destructive agent that is chemically reactive with the active ingredient; and an absorber having the destructive agent impregnated therein. The absorber can be further configured to contact the dermal patch or peel and cause the active ingredient to contact the destructive agent. Such an absorber can be a gauze pad, a sponge or sponge-like material, or the like. In this embodiment, for example, the absorber can be impregnated with a destructive agent or a destructive agent-containing solution. The used dermal patch or peel can be placed onto the absorber such that the active ingredient releasing side of the patch or peel is in contact with the absorber, and thus, capable of becoming in contact with the destructive agent. After a certain amount of time, most, if not all of the active drug in the dermal patch or peel can be destroyed. In one embodiment, the absorber impregnated with the destructive solution can be stored in a closed container to prevent spillage and possible evaporation of any solvent present. That same container can be used for facilitating the contact between the absorber and the dermal patch or peel.

In another embodiment, a method for destroying an active ingredient in a dermal drug delivery device can comprise steps of selecting a destructive agent that is chemically reactive with an active ingredient of a dermal patch or peel; and contacting the destructive agent with the active ingredient, and thus deactivating the active ingredient. Typically, the dermal patch or peel used will be a spent dermal patch or peel.

In the above systems and methods, the active ingredient can be a composition that is to be applied topically or transdermally. However, the invention is particularly adapted to destroying active ingredients that can be harmful if not used or discarded properly, e.g., opiates such as fentanyl, sedatives such as benzodiazepines, hormones, and barbiturates. Examples of destructive agents that can be used to render these and other active ingredients in dermal patches or peels harmless include salts of hypochlorites, potassium permanganate, permanganic acid, salts of permanganic acid, and other oxidants.

In one embodiment, the destructive agent can be substantially dissolved in a solvent to form a destructive agent-containing solvent. This embodiment can provide one of a few advantages. For example, by dissolving a destructive agent sufficiently in a solvent, the destructive agent can diffuse into a dermal patch or peel, and come into contact the active ingredient of the dermal patch or peel. Alternatively, the solvent can be also be configured such that the solvent/destructive agent combination extracts the active ingredient from the dermal patch or peel, thereby increasing contact.

A container, such as a box shaped container or a cylinder shaped container having a resealable lid, such as a press fit lid, screw cap, or plug, can be used. Additionally, in one embodiment, the container can carry an absorbing material therein which is configured to absorb or otherwise be impregnated by the destructive agent. For example, the destructive agent or the destructive agent solution composition can be retained by a thickening agent or even a gel. Such a thickening agent can be used to increase the viscosity of the destructive agent or destructive agent solution such spilling becomes less likely. Alternatively, the destructive agent or the destructive agent solution can be retained in by an absorbent material, such as a sponge or sponge-like material. If a container is used, then the thickening agent or the absorber can be with the destructive agent within the container. However, if the absorber is structurally capable of being self supporting, e.g., a sponge or gauze pad, then a container is not necessary for use.

It can be desirable to destroy certain drugs in dermal delivery systems within an acceptable time. An acceptable time can be determined by considering the intended goals, as well drug to be destroyed. For example, one reason to destroy fentanyl in a fentanyl transdermal delivery patch is to prevent drug abuse. Since a patient may bring a garbage container containing a used fentanyl patch outside for collection within a few hours after disposing of the fentanyl patch, it would be desirable to have an apparatus that is capable of destroying fentanyl within a few hours, or even within a few minutes. The same is true for other drugs having abuse potentials, such as other opiates, sedatives such as benzodiazepines, barbiturates, and the like. On the other hand, the main reason to destroy estradiol in an estradiol transdermal delivery patch is to prevent estradiol from entering the ground water system. Since a closed destruction container likely can survive in a landfill for many months, the destruction of estradiol within a few days or weeks is likely acceptable. In many of the embodiments of the present invention, the destructive agent can be concentrated and configured to destroy at least 80% of the active ingredient in less than two hours. In one embodiment, if the active ingredient is fentanyl, the destructive agent can be concentrated and configured to destroy at least 80% of the fentanyl in less than one to two minutes.

Though the present invention is primarily drawn to the destruction of active ingredients in dermal patches and peels, such as drugs, other ingredients are often also present that can be dangerous to others or the environment. For example, permeation enhancers to facilitate the active ingredient entering or passing the skin or other formulation excipients can also be present. In accordance with embodiments of the present invention, these secondary chemicals that may be present in a dermal patch or peel can alternatively or also be destroyed.

The selection of the destructive agent(s) depends on the particular drug to be destroyed. For example, fentanyl can be readily destroyed by using a solution of sodium hypochlorite. Fentanyl can also be destroyed by potassium permanganate, although typically at a slower rate. Estradiol can be destroyed using sodium hypochlorite as well. Without being bound by a particular theory, it is believed that the mechanism of destruction involving these two agents is most likely oxidation.

Other destructive agents can also be used. For example, light, heat, peroxides, lithium aluminum hydride, trace metals, e.g., iron, cobalt, magnesium, etc., acids, e.g., concentrated or aqueous solutions, bases, e.g., concentrated or aqueous solutions, and/or sodium or potassium metals. Though light and heat are not chemical agents, they can still be considered destructive agents in accordance with embodiments of the present invention.

To provide several specific examples of destructive agent function, multiple destructive schemes are provided. To illustrate, upon light exposure, organic molecules can adsorb energy which is sufficient to dissociate any single bond found in organic molecules. The breaking of these single bonds can result in free radicals which will yield autoxidation products. Metal ions can also initiate autoxidation reactions by direct reaction with substrates in the system. The reaction can involve electron transfer between metal ions and organic compounds. Lithium aluminum hydride reactions can involve the attack of the metal hydride on an electrophilic atom (usually the carbon atom of a carbonyl group) resulting in the reduction of the carbonyl group. Acids and bases can be used as they can aid in catalyzing hydrolysis reactions. Acid and base catalysis can aid in protonating or deprotonating of appropriate functional groups in order to make a reaction possible. Rates of the reaction can be modulated (accelerated or decelerated) by an increase in temperature. Strong acids such as HBr and HI can be used in combination with heat for the cleavage of ethers. These acids are strong enough to protonate the oxygen atom in the ether creating a good leaving group. Halogen ions are also good nucleophiles, which can be used to promote a reaction with a protonated ether. Further, reaction of an active ingredient of a dermal patch or peel with a sodium or potassium metal results in an oxidation-reduction reaction, wherein the metal is oxidized and a hydrogen atom of the drug molecule is reduced. The resulting drug anion can be a good nucleophile which can then react with another reagent creating a therapeutically inactive molecule. An increase in temperature can increase the kinetic energy of the reactant molecules resulting in an increased fraction of the number of molecules with the free energy needed for the reaction to proceed.

In order to destroy an active ingredient or drug, a drug can be brought into a destructive solution or the destructive agent (or solution) can be brought into the drug formulation within the dermal patch or peel, or a combination of both. One method of delivering the destructive agent into the drug formulation is to bring the drug formulation into contact with the destructive solution so that the destructive agent can diffuse into the drug formulation within the dermal patch or peel. For this purpose, the solution of the destructive agent can be configured to have sufficient diffusional driving force into the drug formulation present in the dermal patch or peel. A “sufficient diffusional driving force” can be defined as a diffusional driving force that is high enough to drive sufficient amount of the destructive agent into the drug formulation within the dermal patch or peel to destroy most or all of the drug within an acceptable period of time. For example, it can be desirable that the solvent system for the destructive agent is made such that the destructive agent has lower solubility in its solution than in the drug formulation. Thus, the destructive agent can partition favorably into the drug formulation. Otherwise, if the solubility of the destructive agent in its solution is much higher than that in the drug formulation, the destructive agent may tend to stay in the solution rather than to go into the drug formulation. With this latter arrangement, the destruction can take a prolonged amount of time, which can be undesirable for situations where the prevention of drug abuse is desired. This being stated, slower destruction is still considered to be within the scope of the present invention. On the other hand, a destructive solution can be configured to have reasonable solubility of the destructive agent within the solvent. As such, one aspect of this invention is related to a solution comprising a destructive agent, and a solution designed so that the solubility of the destructive agent is low enough for efficient destructive agent permeation into the drug formulation, while being high enough for solubilizing sufficient quantities of the destructive agent for the satisfactory destruction of the drug.

As mentioned earlier, the destruction of the drug can also occur if the drug is extracted out of the drug formulation and into the destructive solution. Generally, this can be accomplished if the drug has a high solubility in the destructive solution so that it tends to leave the dermal patch or peel in favor of contacting the destructive solution. Again, this can be achieved by manipulating the properties of the destructive solution. For example, if the drug to be destroyed is a base, the destructive solution can generally be made to be acidic. This is because basic drugs usually have higher solubility in acidic solutions, and hence, a higher tendency to leave the formulation and enter the destructive solution. Another aspect of the present invention is related to a solution comprising a destructive agent, and further, the solution is configured such that the solubility of the drug to be destroyed high enough to efficiently extract the drug out of the formulation. It is also possible that a practical apparatus is configured to destroy the drug by using the combination of the above two mechanisms.

Turning to embodiments where the drug is not necessarily destroyed, but is rendered substantially inaccessible, systems and methods for impeding the unintended use of residual active ingredients that may be present in a dermal drug delivery device are provided. In one embodiment, such a system can comprise a dermal patch including a first side configured to deliver an active ingredient to a skin or mucosal surface, and a device including a glue-coated surface. The first side of the dermal patch can also include a dermal adhesive, and the glue-coated surface of the device can be configured to adhere to the first side. Thus, upon contacting the glue-coated surface with the first side, residual active ingredient can be rendered substantially inaccessible. Typically, the substrate of the device can be impermeable to the drug.

In another embodiment, a method for impeding the unintended use of residual active ingredients present in a dermal drug delivery device can comprise steps of obtaining a dermal patch including first side configured to deliver an active ingredient to a skin or mucosal surface, and adhering the first side of the spent dermal patch to a glue-coated surface of a device, thereby rendering residual active ingredient substantially inaccessible. In one embodiment, the first side of the dermal patch includes a dermal adhesive. Typically, the spent dermal patch can include residual active ingredient after use.

In both the system and method relating to impeding the unintended use of an active ingredient set forth above, the adhesive-coated surface of the device or substrate can be configured to bond with the dermal adhesive such that an increase in the bonding strength between the dermal patch and the device is realized. In other words, though not required, the dermal adhesive and the adhesive-coated surface of the device can work synergistically to improve bonding strength. Additionally, in one embodiment, the adhesive-coated device can also include a destructive agent that is configured to contact the active ingredient. If the destructive agent is not present with the adhesive-coated layer of the device, it can be present in a layer wherein the destructive agent or the active ingredient is allowed to pass through the adhesive-coated layer so that the destructive agent and the active ingredient come in contact. In yet another embodiment, the device can be substantially impermeable to the active ingredient.

In related embodiments, a system for impeding unintended use of residual active ingredients present in a dermal patch or peel can comprise a dermal patch or peel configured to deliver an active ingredient to a skin or mucosal surface; and a device configured to envelop and seal the dermal patch or peel therein such that the active ingredient is rendered substantially inaccessible for unintended use. Further, a method for impeding the unintended use of residual active ingredients present in a dermal patch or peel can comprise steps of obtaining a dermal patch or peel configured to deliver an active ingredient to a skin or mucosal surface; and sealing the dermal patch or peel within the device, thereby rendering the active ingredient substantially inaccessible for unintended use.

Though applicable to both dermal peels and patches, as there is no backing on a dermal peel, active ingredient can be accessible from either side of the peel. Thus, dermal peels particularly would benefit from a device that can enfold or enclose the peel. Such enclosing or enfolding can be carried out by the use of a glue-coated device that is folded over the dermal peel such that the peel becomes sealed within the folded device, for example. Alternatively, two devices, each having glue-coated surfaces, can be used to sandwich and seal the dermal peel therein, as would be know by one skilled in the art after considering the present disclosure.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention.

### Example 1

### Fentanyl Destruction in Sodium Hypochlorite Solution

About 3 mg of fentanyl and 2.8 mg of citric acid is dissolved in 5 mL of water. After, the fentanyl is dissolved in the solution, 5 mL of a concentrated sodium hypochlorite solution (<0.2 wt % sodium hydroxide; 6-9 wt % sodium hypochlorite) is added to the aqueous solution. The aqueous solution is then placed on an orbital shaker set at 150 rpm. At predetermined time points, aliquots of the solution are removed and the amount of fentanyl remaining at each time interval is determined using HPLC, as illustrated in Table 1 below:

TABLE 1
Fentanyl degradation as a function of time

| Time (hrs) | Fentanyl (mg in 10 mL solution) | % Degraded |
|---|---|---|
| 0 | 3.50 | |
| 1 | 2.34 | 33 |
| 2 | 1.57 | 55 |
| 4 | 0.34 | 90 |
| 8 | 0.00 | 100 |

As illustrated in Table 1 above, over 90% of the fentanyl is degraded within 4 hours when placed in the hypochlorite solution prepared in accordance with the present example.

### Example 2

### Estradiol Destruction in Acetonitrile/Sodium Hypochlorite Solution

About 2 mg of estradiol is dissolved in 2.5 mL acetonitrile. To this solution is added 7.5 mL of a concentrated sodium hypochlorite solution (<0.2 wt % sodium hydroxide; 6-9 wt % sodium hypochlorite). The aqueous solution is then placed on an orbital shaker set at 150 rpm. At predetermined time points, aliquots are removed and the amount of estradiol is determined using HPLC. The earliest time point (˜5 sec) shows that the estradiol is from substantially to completely destroyed.

### Example 3

### Clonazepam Destruction in Sodium Hypochlorite Solution

About 1 mg of clonazepam is dissolved in 0.5 mL acetonitrile. To this solution is added 3 mL of a concentrated sodium hypochlorite solution (<0.2 wt % sodium hydroxide; 6-9 wt % sodium hypochlorite). Immediately upon addition of the sodium hypochlorite solution, the solution turns a dark yellow and after 9-18 minutes solution gradually turns a lighter yellow. The aqueous solution is then placed on an orbital shaker set at 150 rpm. At predetermined time points, aliquots are removed and the amount of clonazepam is determined using HPLC as illustrated in Table 2 below:

TABLE 2
Clonazepam degradation as a function of time

| | Clonazepam (mg in | |
|---|---|---|
| Time (min) | 10 mL solution) | % Degraded |
| 0 | 0.86 | |
| 1 | 0.52 | 40 |
| 3 | 0.25 | 71 |
| 9 | 0.03 | 97 |
| 27 | 0.00 | 100 |

As illustrated in Table 2 above, over 90% of the fentanyl is degraded within 9 minutes when placed in the hypochlorite solution prepared in accordance with the present example.

### Example 4

### Fentanyl Destruction by Potassium Permanganate

A 2.17 mg amount of fentanyl and 2.8 mg of citric acid is dissolved in 10 mL water. Next, 17 mg of potassium permanganate is added to the aqueous solution. The aqueous solution is placed on an orbital shaker set at 150 rpm. At predetermined time points, aliquots are removed and the amount of fentanyl remaining is determined using HPLC, the results being set forth in Table 3 below:

TABLE 3
Fentanyl degradation as a function of time

| | Fentanyl (mg in | |
|---|---|---|
| Time (min) | 10 mL solution) | % Degraded |
| 0 | 2.17 | |
| 10 | 1.64 | 24 |
| 20 | 0.57 | 74 |
| 40 | 0.06 | 97 |
| 60 | 0.02 | 99 |

As illustrated in Table 3 above, over 90% of the fentanyl is degraded within 40 minutes. Once the pharmaceutical agent is in solution, the rate of degradation proceeds more rapidly.

### Example 5

### Fentanyl Destruction in Sodium Hypochlorite Solution Using a Sponge

A re-sealable, flat box having inner dimensions of about 10×15×0.3 cm is fitted with a spongy material having an outer dimension that substantially matches the inner dimension of the box. The flat box has a press-fit lid along one of its 10×15 cm surfaces that can be closed and substantially sealed due to the presence of a rectangular rubber ring at the interface between the box and the lid. About 35 mL of a 6-9 wt % sodium hypochlorite in water solution is impregnated into the spongy material. After absorbing the sodium hypochlorite solution into the spongy material, the drug releasing side of a transdermal fentanyl patch is placed in contact with the spongy material saturated with the sodium hypochlorite solution. The lid is then closed. In the present example, most of the fentanyl in the patch is destroyed within one hour. The patch can remain in the box over a long period of time without adverse affects, e.g., until the box is opened to replace the destroyed fentanyl patch with another spent fentanyl patch. After three days, the patch is removed from the box, and is replaced with another patch. Similarly, after about an hour, most of the fentanyl in the patch is also destroyed.

### Example 6

### Fentanyl Destruction in Sodium Hypochlorite Solution in Flat Box

A substantially rectangular container with inner dimensions of about 10×15×0.3 cm, and a slit opening on one of the 10×0.3 cm sides (the top side) is provided. The slit opening has dimensions of 8×0.2 cm, and can be covered by a sealable lid or plug. About 35 mL of 6-9 wt % sodium hypochlorite solution is placed into the container. A fentanyl patch is dropped into the container via the slit opening, and the lid is used to seal the opening. Within one hour, most of the fentanyl within the patch that is submersed in the hypochlorite solution is destroyed. In this configuration, multiple spent fentanyl patches can be dropped into the box, resulting in multiple patches being substantially devoid of fentanyl.

### Example 7

### Fentanyl Destruction in Sodium Hypochlorite Solution in Cylindrical container

Example 6 was repeated, except that the container used was cylindrical with an opening capable of being capped with a screw cap. A fentanyl patch is rolled tightly and dropped into the container via the small round opening, and the lid is screwed on to seal the opening. Within one hour, most of the fentanyl within the patch that is submersed in the hypochlorite solution is destroyed. In this configuration, multiple spent fentanyl patches can rolled up and be dropped into the cylinder, resulting in multiple patches being substantially devoid of fentanyl.

### Example 8

### Fentanyl Patch Rendered Harmless Using Substrate Adhesive Sheet

A backing film with dimensions of 10×15 cm is coated with a 0.3 cm layer of a substrate adhesive to form an adhesive sheet. The substrate adhesive layer of the adhesive sheet is covered with a release liner. After removing the release liner, a fentanyl patch is adhered to the adhesive sheet such that the drug releasing side of the fentanyl patch is adhered to the device, which includes the substrate adhesive layer of the adhesive sheet. The bond between patch and adhesive sheet is resistant to peel, sheer, and tensile stress, as well as being resistant to exposure to water and chemical solvents.

While the invention has been described with reference to certain preferred embodiments, those skilled in the art will appreciate that various modifications, changes, omissions, and substitutions can be made without departing from the spirit of the invention.

1. A system for reducing unintended use of an active ingredient, comprising:
a dermal patch or peel including the active ingredient;
a destructive agent that is chemically reactive with the active ingredient; and
a device for supporting the destructive agent, said device being configured to receive the dermal patch or peel and enable the active ingredient to contact the destructive agent.

2. The system of claim 1, wherein the device is in the form of a container that contains the destructive agent, wherein the container is also configured to receive the dermal patch or peel within the container.

3. The system of claim 1, wherein the active ingredient is a member selected from the group consisting of a CNS depressant, an opiate, a sedative, a hormone, and a barbiturate.

4. The system of claim 1, wherein the active ingredient is an opiate, said opiate being fentanyl.

5. The system of claim 1, wherein the active ingredient is a sedative, said sedative being a benzodiazepine.

6. The system of claim 1, wherein the destructive agent is selected from the group consisting salts of hypochlorites, potassium permanganate, permanganic acid, and salts of permanganic acid.

7. The system of claim 1, wherein the destructive agent is substantially dissolved in a solvent to form a destructive agent solution composition.

8. The system of claim 7, wherein the destructive agent solution composition is retained at said device by a thickening agent.

9. The system of claim 7, wherein the destructive agent solution composition is retained at said device by an absorbent material.

10. The system of claim 1, wherein the destructive agent is configured to destroy at least 80% of said active ingredient in less than 24 hours.

11. The system of claim 1, wherein the destructive agent is configured to destroy at least 80% of said active ingredient in less than 12 hours.

12. The system of claim 1, wherein the destructive agent is configured to destroy at least 80% of said active ingredient in less than 2 hours.

13. The system of claim 1, wherein the active ingredient is fentanyl, and the destructive agent is a solution of sodium hypochloride configured to destroy at least 80% of the fentanyl in less than 20 minutes.

14. The system of claim 2, wherein the container has an opening capable of being tightly closed with a press fit lid, screw cap, or plug.

15. The system of claim 7, wherein the destructive agent is sufficiently soluble in the solvent such that the destructive agent is diffusible into the dermal patch or peel, thereby contacting the active ingredient.

16. The system of claim 7, wherein said solvent is configured to extract the active ingredient from the dermal patch or peel, thereby contacting the active ingredient.

17. The system of claim 1, wherein the dermal patch or peel is spent.

18. The system of claim 1, wherein the dermal patch or peel is a transdermal delivery device.

19. A system for reducing unintended use of an active ingredient, comprising:
a dermal patch or peel including the active ingredient;
a destructive agent that is chemically reactive with the active ingredient; and
an absorber having the destructive agent impregnated therein, said absorber being further configured to contact the dermal patch or peel and enable the active ingredient to contact the destructive agent.

20. The system of claim 19, wherein the absorber is a spongy material.

21. The system of claim 19, wherein the absorber is carried by a substrate.

22. The system of claim 21, wherein the substrate is in the form of a container that contains the absorber, wherein the container is also configured to receive the dermal patch or peel within the container where the dermal patch or peel contacts the absorber.

23. The system of claim 19, wherein the active ingredient is a member selected from the group consisting of an opiate, a sedative, a hormone, and a barbiturate.

24. The system of claim 23, wherein the active ingredient is the opiate, said opiate being fentanyl.

25. The system of claim 23, wherein the active ingredient is the sedative, said sedative being a benzodiazepine.

26. The system of claim 19, wherein the destructive agent is an oxidant selected from the group consisting salts of hypochlorites, potassium permanganate, permanganic acid, and salts of permanganic acid.

27. The system of claim 19, wherein the destructive agent is substantially dissolved in a solvent to form a destructive agent solution composition.

28. The system of claim 19, wherein the destructive agent is configured to destroy at least 80% of said active ingredient in less than 24 hours.

29. The system of claim 19, wherein the destructive agent is configured to destroy at least 80% of said active ingredient in less than 12 hours.

30. The system of claim 19, wherein the destructive agent is configured to destroy at least 80% of said active ingredient in less than 2 hours.

31. The system of claim 19, wherein the active ingredient is fentanyl, and the destructive agent is a solution of sodium hypochloride configured to destroy at least 80% of the fentanyl in less than 24 hours.

32. The system of claim 22, wherein the container has an opening capable of being tightly closed with a press fit lid, screw cap, or plug.

33. The system of claim 27, wherein the destructive agent is sufficiently soluble in the solvent such that the destructive agent is diffusible into the dermal patch or peel, thereby contacting the active ingredient.

34. The system of claim 27, wherein said solvent is configured to extract the active ingredient from the dermal patch or peel, thereby contacting the active ingredient.

35. The system of claim 19, wherein the dermal patch or peel is a spent dermal delivery device.

36. The system of claim 19, wherein the dermal patch or peel is a transdermal delivery device.

37. A method for destroying an active ingredient in a dermal drug delivery device, comprising:
selecting a destructive agent that is chemically reactive with an active ingredient of a dermal patch or peel; and
contacting the destructive agent with the active ingredient, and thus deactivating the active ingredient.

38. A method as in claim 37, wherein the contacting occurs in a container.

39. A method as in claim 37, further comprising the step of impregnating an absorber with the destructive agent, wherein the contacting step occurs by contacting the active ingredient with the impregnated absorber.

40. A method as in claim 39, wherein the absorber is carried by a substrate.

41. A method as in claim 40, wherein the substrate is in the form of a container that contains the absorber, wherein the container is also configured to receive the dermal patch or peel within the container where the dermal patch or peel contacts the absorber.

42. A method as in claim 37, wherein the active ingredient is a member selected from the group consisting of an opiate, a sedative, a hormone, and a barbiturate.

43. A method as in claim 42, wherein the active ingredient is the opiate, said opiate being fentanyl.

44. A method as in claim 42, wherein the active ingredient is the sedative, said sedative being a benzodiazepine.

45. A method as in claim 37, wherein the destructive agent is an oxidant selected from the group consisting salts of hypochlorites, potassium permanganate, permanganic acid, and salts of permanganic acid.

46. A method as in claim 37, wherein the destructive agent is substantially dissolved in a solvent to form a destructive agent solution composition.

47. A method as in claim 37, wherein the destructive agent is configured to destroy at least 80% of said active ingredient in less than 24 hours.

48. A method as in claim 37, wherein the destructive agent is configured to destroy at least 80% of said active ingredient in less than 12 hours.

49. A method as in claim 37, wherein the destructive agent is configured to destroy at least 80% of said active ingredient in less than 2 hours.

50. A method as in claim 37, wherein the active ingredient is fentanyl, and the destructive agent is a solution of sodium hypochloride configured to destroy at least 80% of the fentanyl in less than 24 hours.

51. A method as in claim 46, wherein the destructive agent is sufficiently soluble in the solvent such that the destructive agent is diffusible into the dermal patch or peel, thereby contacting the active ingredient.

52. A method as in claim 46, wherein said solvent is configured to extract the active ingredient from the dermal patch or peel, thereby contacting the active ingredient.

53. A method as in claim 37, wherein the dermal patch or peel is a spent dermal delivery device.

54. A method as in claim 37, wherein the dermal patch or peel is a transdermal delivery device.

55. A system for impeding the unintended use of an active ingredient, comprising:
a dermal patch including a first side configured to deliver the active ingredient to a skin or mucosal surface, said first side also including a dermal adhesive; and
a device including a glue-coated surface, said glue-coated surface being configured to adhere to the first side, wherein upon contacting the glue-coated surface with the first side, the active ingredient is rendered substantially inaccessible.

56. A system as in claim 55, wherein the glue-coated surface is configured to bond with the first side of the dermal patch to provide bonding between the dermal patch and the device.

57. A system as in claim 55, wherein glue-coated surface includes a destructive agent.

58. A system as in claim 57, wherein the active ingredient is selected from the group consisting of opiates, sedatives, hormones, and barbiturates.

59. A system as in claim 58, wherein the active ingredient is the opiate, said opiate being fentanyl.

60. A system as in claim 58, wherein the active ingredient is the sedative, said sedative being a benzodiazepine.

61. A system as in claim 55, wherein the device is impermeable to the active ingredient.

62. A system as in claim 55, wherein the dermal patch is a spent dermal patch.

63. A system as in claim 55, wherein the dermal patch is a transdermal patch.

64. A method for impeding the unintended use of an active ingredient, comprising:
obtaining a dermal patch including a first side configured to deliver the active ingredient to a skin or mucosal surface, said first side also including a dermal adhesive; and
adhering the first side of the dermal patch to a glue-coated surface of a device, thereby rendering the active ingredient substantially inaccessible.

65. A method as in claim 64, wherein the glue-coated surface is configured to bond with the first side of the dermal patch to provide bonding between the dermal patch and the device.

66. A method as in claim 64, wherein glue-coated surface includes a destructive agent.

67. A method as in claim 66, wherein the active ingredient is selected from the group consisting of opiates, sedatives, hormones, and barbiturates.

68. A method as in claim 67, wherein the active ingredient is the opiate, said opiate being fentanyl.

69. A method as in claim 67, wherein the active ingredient is the sedative, said sedative being a benzodiazepine.

70. A method as in claim 64, wherein the device is impermeable to the active ingredient.

71. A method as in claim 64, wherein the dermal patch is a spent dermal patch.

72. A method as in claim 64, wherein the dermal patch is a transdermal patch.

73. A system for impeding unintended use of an active ingredient, comprising:
a dermal patch or peel configured to deliver the active ingredient to a skin or mucosal surface; and
a device configured to envelop and seal the dermal patch or peel therein such that the active ingredient is rendered substantially inaccessible for unintended use.

74. A system as in claim 73, wherein the device includes a glue-coated surface and the glue-coated surface is used seal the dermal patch or peel therein.

75. A system as in claim 74, wherein the device is configured to be folded around the dermal patch or peel.

76. A system as in claim 74, wherein glue-coated surface includes a destructive agent.

77. A system as in claim 73, wherein the active ingredient is selected from the group consisting of opiates, sedatives, hormones, and barbiturates.

78. A system as in claim 77, wherein the active ingredient is the opiate, said opiate being fentanyl.

79. A system as in claim 77, wherein the active ingredient is the sedative, said sedative being a benzodiazepine.

80. A method for impeding the unintended use of an active ingredient, comprising:
obtaining a dermal patch or peel configured to deliver the active ingredient to a skin or mucosal surface; and
sealing the dermal patch or peel within the device, thereby rendering the active ingredient substantially inaccessible for unintended use.

81. A method as in claim 80, wherein the device includes a glue-coated surface and the glue-coated surface is used seal the dermal patch or peel therein.

82. A system as in claim 81, wherein the step of sealing includes enveloping the dermal patch or peel with the device.

83. A system as in claim 81, wherein the device is configured to be folded around the dermal patch or peel.

84. A system as in claim 81, wherein glue-coated surface includes a destructive agent.

85. A system as in claim 80, wherein the active ingredient is selected from the group consisting of opiates, sedatives, hormones, and barbiturates.

86. A system as in claim 80, wherein the active ingredient is the opiate, said opiate being fentanyl.

87. A system as in claim 80, wherein the active ingredient is the sedative, said sedative being a benzodiazepine.
